(19) United States
(12) Patent Application Publication
(10) Pub. No.: US 2025/0084125 A1
(43) Pub. Date: Mar. 13, 2025

(54) HIGH VISCOSITY ULTRAFILTRATION/DIAFILTRATION AND SINGLE-PASS TANGENTIAL FLOW FILTRATION PROCESSES

(71) Applicant: Genentech, Inc., South San Francisco, CA (US)

(72) Inventors: Benjamin Mark SACKETT, San Francisco, CA (US); James J. KEBA, Pacifica, CA (US)

(73) Assignee: Genentech, Inc., South San Francisco, CA (US)

(21) Appl. No.: 18/826,814

(22) Filed: Sep. 6, 2024

Related U.S. Application Data
(60) Provisional application No. 63/581,474, filed on Sep. 8, 2023.

(51) Int. Cl. C07K 1/34; B01D 61/14; B01D 69/02

(52) U.S. Cl. CPC C07K 1/34; B01D 61/146; B01D 69/02; B01D 2315/10; B01D 2315/16; B01D 2317/02; B01D 2317/04; B01D 2325/34

(57) ABSTRACT

The present disclosure provides high viscosity ultrafiltration/diafiltration (UF/DF) and single-pass tangential flow filtration (SPTFF) processes used in the purification of polypeptides. A method for purifying a polypeptide from a polypeptide preparation may include the following steps in order: a) subjecting the polypeptide preparation to one or more purification processes and recovering a first resulting pool having a viscosity of about 5-40 cP; and b) subjecting the pool recovered from step a) to a SPTFF operation comprising use of membranes in either or both a serial membrane and/or a parallel membrane configuration and recovering a second resulting pool having an operating viscosity of about 41-400 cP, wherein, i) the operating temperature is about 15-55° C., and ii) the feed flux is about 5-50 L/m²/hr (LMH).

### CROSS REFERENCE TO RELATED APPLICATIONS

This application claims priority to U.S. Provisional Patent Application Ser. No. 63/581,474, filed Sep. 8, 2023, the entire contents of which are incorporated herein by reference.

### FIELD

The present disclosure provides high viscosity ultrafiltration/diafiltration (UF/DF) and single-pass tangential flow filtration (SPTFF) processes used in the purification of polypeptides.

### BACKGROUND

Single-Pass Tangential Flow Filtration (SPTFF) enables new technologies and advances in protein purification to support increased productivity across the biotherapeutic manufacturing industry. SPTFF systems differ from traditional UFDF systems in reduced membrane area, smaller process volumes, and shorter operating time. It may also differ in operating temperature, and the application of multiple stages of ultrafiltration. Further, the initial process concentration is relevant to process control, where typical multi-pass UFDF operations are minimally affected by initial concentration. SPTFF increases protein solution concentration by removing buffer solution as it flows across a semipermeable membrane while retaining product. Protein concentration is a key process parameter which dictates ocular and subcutaneous dosing capabilities. However, viscosity increases exponentially as protein concentration increases which results in high pressures in the feed channel and low flux at the membrane boundary. High viscosity therefore limits the maximum achievable protein concentration in TFF based ultrafiltration.

All references cited herein, including patent applications and publications, are incorporated by reference in their entirety.

### BRIEF SUMMARY

An SPTFF process, developed under operating conditions which minimize feed channel pressure and operating viscosity, is ideal for achieving maximum protein concentration. Disclosed herein are systems and methods for high viscosity ultrafiltration/diafiltration (UF/DF) and SPTFF processes used in the purification of polypeptides. The systems and methods disclosed herein can include one or more purification processes followed by an SPTFF operation. The one or more purification processes can include ultrafiltration and diafiltration. The one or more purification processes can result in a pool having a viscosity of between about 5-40 cP, which can then be subjected to an SPTFF operation. The SPTFF operation can include using membranes in either or both of a serial and/or parallel configuration to produce a resulting pool having an operating viscosity of about 41-400 cP.

According to an aspect, a method for purifying a polypeptide from a polypeptide preparation, comprises the following steps in order: a) subjecting the polypeptide preparation to one or more purification processes and recovering a first resulting pool having a viscosity of about 5-40 cP; and b) subjecting the pool recovered from step a) to a single pass tangential flow filtration (SPTFF) operation and recovering a second resulting pool having an operating viscosity of about 41-400 cP, wherein, i) the operating temperature is about 15-55° C., and ii) the feed flux is about 5-50 L/m²/hr (LMH).

In some embodiments of the method, in step a) the viscosity of the first recovered pool is 10-35 cP.

In some embodiments of the method, in step b) the STPFF operation comprises use of a serial membrane configuration.

In some embodiments of the method, in step b) the STPFF operation comprises use of a parallel membrane configuration.

In some embodiments of the method, in step b) the SPTFF operation comprises use of both a serial membrane configuration and a parallel membrane configuration.

In some embodiments of the method, in step b) wherein the membrane configuration is any of 1:1:1, 1:1:1:1, 2:1:1, or 2:2:1:1.

In some embodiments of the method, in step b) the membrane configuration comprises at least one of 10 kD membranes and 30 kD membrane.

In some embodiments of the method, in step b) the membrane configuration comprises at least one of narrow channel membranes and wide channel membranes.

In some embodiments of the method, in step a) the polypeptide preparation is formulated in a viscosity-reducing excipient.

In some embodiments of the method, in step b) the operating temperature is about 15-50° C. or about 30-50° C.

In some embodiments of the method, in step b) the feed flux is about 5-25 LMH or about 25-50 LMH.

In some embodiments of the method, the operating viscosity in step b) is about 200-400 cP or about 200-300 cP.

In some embodiments of the method, in step b) the retentate pressure is about 5-20 psi.

In some embodiments of the method, the one or more purification processes of step a) comprises an ultrafiltration.

In some embodiments of the method, the one or more purification processes of step a) comprises at least two ultrafiltration operations.

In some embodiments of the method, the one or more purification processes of step a) comprises a diafiltration.

In some embodiments of the method, step a) comprises i) subjecting the polypeptide preparation to an ultrafiltration operation to a first target concentration, ii) subjecting the polypeptide preparation to a diafiltration operation for buffer exchange, and iii) subjecting the polypeptide preparation to a second ultrafiltration operation to a second target concentration and recovering the first resulting pool having a viscosity of 5-40 cP.

In some embodiments of the method, the first target concentration is 5-80 g/L.

In some embodiments of the method, the buffer exchange is a 6-12× exchange.

In some embodiments of the method, the second target concentration is 100-150 g/L or 100-200 g/L.

In some embodiments of the method, the polypeptide preparation comprises the polypeptide and one or more impurities.

In some embodiments of the method, the one or more purification processes of step a) comprise at least one of: a harvest step, a sample conditioning step, a viral filtration step, a viral inactivation step, a chromatography polishing step, a chromatography capture step, a dilution step, a tangential flow depth filtration step, a depth filtration step, a diafiltration step, an ultrafiltration step, a guard filtration step, a precipitation step, and a flocculation step.

In some embodiments, the method further comprises: subjecting the second resulting pool to one or more subsequent purification processes, wherein the one or more subsequent purification processes comprise at least one of: a viral filtration step, a viral inactivation step, a chromatography polishing step, a chromatography capture step, a dilution step, a sample conditioning step, a diafiltration step, a tangential flow depth filtration step, a depth filtration step, a sterile filtration step, and a guard filtration step.

According to an aspect, a system for purifying a polypeptide from a polypeptide preparation, the system configured to perform the following steps in order: a) subject the polypeptide preparation to one or more purification processes and recover a first resulting pool having a viscosity of about 5-40 cP; and b) subject the pool recovered from step a) to a single pass tangential flow filtration (SPTFF) operation and recover a second resulting pool having an operating viscosity of about 41-400 cP, wherein, i) the operating temperature is about 15-55° C., and ii) the feed flux is about 5-50 L/m²/hr (LMH).

In some embodiments of the system, in step a) the viscosity of the first recovered pool is 10-35 cP.

In some embodiments of the system, in step b) the STPFF operation comprises use of a serial membrane configuration.

In some embodiments of the system, in step b) the STPFF operation comprises use of a parallel membrane configuration.

In some embodiments of the system, in step b) the SPTFF operation comprises use of both a serial membrane configuration and a parallel membrane configuration.

In some embodiments of the system, in step b) wherein the membrane configuration is any of 1:1:1, 1:1:1:1, 2:1:1, or 2:2:1:1.

In some embodiments of the system, in step b) the membrane configuration comprises at least one of 10 kD membranes and 30 kD membrane.

In some embodiments of the system, in step b) the membrane configuration comprises at least one of narrow channel membranes and wide channel membranes.

In some embodiments of the system, in step a) the polypeptide preparation is formulated in a viscosity-reducing excipient.

In some embodiments of the system, in step b) the operating temperature is about 15-50° C. or about 30-50° C.

In some embodiments of the system, in step b) the feed flux is about 5-25 LMH or about 25-50 LMH.

In some embodiments of the system, the operating viscosity in step b) is about 200-400 cP or about 200-300 cP.

In some embodiments of the system, in step b) the retentate pressure is about 5-20 psi.

In some embodiments of the system, the one or more purification processes of step a) comprises an ultrafiltration.

In some embodiments of the system, the one or more purification processes of step a) comprises at least two ultrafiltration operations.

In some embodiments of the system, the one or more purification processes of step a) comprises a diafiltration.

In some embodiments of the system, in step a) the system is configured to: i) subject the polypeptide preparation to an ultrafiltration operation to a first target concentration, ii) subject the polypeptide preparation to a diafiltration operation for buffer exchange, and iii) subject the polypeptide preparation to a second ultrafiltration operation to a second target concentration and recover the first resulting pool having a viscosity of 5-40 cP.

In some embodiments of the system, the first target concentration is 5-80 g/L.

In some embodiments of the system, the buffer exchange is a 6-12× exchange.

In some embodiments of the system, the second target concentration is 100-150 g/L or 100-200 g/L.

In some embodiments of the system, the polypeptide preparation comprises the polypeptide and one or more impurities.

In some embodiments of the system, the one or more purification processes of step a) comprise at least one of: a harvest step, a sample conditioning step, a viral filtration step, a viral inactivation step, a chromatography polishing step, a chromatography capture step, a dilution step, a tangential flow depth filtration step, a depth filtration step, a diafiltration step, an ultrafiltration step, a guard filtration step, a precipitation step, and a flocculation step.

In some embodiments of the system, the system is further configured to: subject the second resulting pool to one or more subsequent purification processes, wherein the one or more subsequent purification processes comprise at least one of: a viral filtration step, a viral inactivation step, a chromatography polishing step, a chromatography capture step, a dilution step, a sample conditioning step, a diafiltration step, a tangential flow depth filtration step, a depth filtration step, a sterile filtration step, and a guard filtration step.

### BRIEF DESCRIPTION OF THE DRAWINGS

FIGS. 1A and 1B show the retentate concentration at different feed fluxes for three different feed concentrations (140 g/L, 155 g/L and 170 g/L). Operating temperatures of 50° C. (FIG. 1A) and 40° C. (FIG. 1B) were evaluated.

FIG. 1C shows the relationship between feed pressure and feed flux at different feed concentrations and operating temperatures.

FIGS. 1D and 1E show the viscosities of an antibody preparation at different feed concentrations and temperatures.

FIG. 2 shows retentate concentrations and feed pressures at different feed fluxes of an antibody passed through a SPTFF operation at 40° C.

FIG. 3 shows an exemplary computing device which may be implemented in accordance with one or more examples of the disclosure.

### DETAILED DESCRIPTION

In some aspects, systems and methods are provided for purifying a polypeptide from a polypeptide preparation, comprising the following steps in order: a) subjecting the polypeptide preparation to one or more purification processes and recovering a first resulting pool having a viscosity of about 5-40 cP; b) subjecting the pool recovered from step a) to an SPTFF operation comprising use of membranes in either or both of a serial membrane configuration and/or a parallel membrane configuration and recovering the resulting pool having an operating viscosity of about 41-400 cP wherein, i) the operating temperature is about 15-55° C., ii) the feed flux is about 5-50 L/m²/hr (LMH). In some embodiments, the one or more purification processes is an ultrafiltration process; for example, an ultrafiltration/diafiltration/ultrafiltration process.

It will also be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects.

### Definitions

For purposes of interpreting this specification, the following definitions will apply and, whenever appropriate, terms used in the singular will also include the plural and vice versa. In the event that any definition set forth below conflicts with any document incorporated herein by reference, the definition set forth shall control.

The term “polypeptide” or “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. The terms “polypeptide” and “protein” as used herein specifically encompass antibodies.

“Purified” polypeptide (e.g., antibody or immunoadhesin) means that the polypeptide has been increased in purity, such that it exists in a form that is more pure than it exists in its natural environment and/or when initially synthesized and/or amplified under laboratory conditions. Purity is a relative term and does not necessarily mean absolute purity.

The term “antibody” includes full-length antibodies and antigen-binding fragments thereof. In some embodiments, a full-length antibody comprises two heavy chains and two light chains. The variable regions of the light and heavy chains are responsible for antigen binding. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani et al., 1997, JMB, 273: 927-948; Chothia 1985; Chothia 1987; Chothia 1989; Kabat 1987; Kabat 1991), or by the AbM method (see, e.g., Martin et al., PNAS, 1989, 86(23): 9268-9272), IMGT method (see, e.g., Lefranc et al., Dev Comp Immunol, 2003, 27(1): 55-77), or Contact method (see, e.g., MacCallum et al., J. Mol. Biol., 1996, 262: 732-745), or other known schemes. The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as lgG1 (γ1 heavy chain), lgG2 (γ2 heavy chain), lgG3 (γ3 heavy chain), lgG4 (γ4 heavy chain), lgA1 (α1 heavy chain), or lgA2 (α2 heavy chain). In some embodiments, the antibody is a chimeric antibody. In some embodiments, the antibody is a semi-synthetic antibody. In some embodiments, the antibody is a diabody. In some embodiments, the antibody is a humanized antibody. In some embodiments, the antibody is a multispecific antibody, such as a bispecific antibody. In some embodiments, the antibody is linked to a fusion protein. In some embodiments the antibody is linked to an immunostimulating protein, such as an interleukin. In some embodiments the antibody is linked to a protein which facilitates the entry across the blood brain barrier. In some embodiments, the antibody is linked to a small molecule drug, for instance, ADC.

The term “antigen-binding fragment” as used herein refers to an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

The term “chimeric antibodies” refer to antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit a biological activity of this invention (see U.S. Pat. No. 4,816,567; and Morrison et al., Proc. Natd. Acad. Sci. USA, 81:6851-6855 (1984)).

The term “multispecific antibodies” as used herein refer to monoclonal antibodies that have binding specificities for at least two different sites, i.e., different epitopes on different antigens or different epitopes on the same antigen. In certain aspects, the multispecific antibody has two binding specificities (bispecific antibody). In certain aspects, the multispecific antibody has three or more binding specificities. Multispecific antibodies may be prepared as full length antibodies or antibody fragments.

The term “semi-synthetic” in reference to an antibody or antibody means that the antibody or antibody has one or more naturally occurring sequences and one or more non-naturally occurring (i.e., synthetic) sequences.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv,” also abbreviated as “sFv” or “scFv,” are antibody fragments that comprise the V_{H} and V_{L} antibody domains connected into a single polypeptide chain. In some embodiments, the scFv polypeptide further comprises a polypeptide linker between the V_{H} and V_{L} domains which enables the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing scFv fragments (see preceding paragraph) typically with short linkers (such as about 5 to about 10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” scFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region (HVR) of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “sequential” as used herein with regard to purification processes refers to having a first purification process followed by a second purification process. Additional steps may be included between the first purification process and the second purification process.

The term “continuous” as used herein with regard to purification processes refers to having a first purification process and a second purification process either directly connected or comprising some other mechanism which allows for continuous flow between the two purification processes.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated”.

“Contaminants” refer to materials that are different from the desired polypeptide product. The contaminant includes, without limitation: host cell materials, such as CHO host cell protein (CHOP); leached Protein A; nucleic acid; a variant (e.g., a basic variant or an acidic variant of the desired polypeptide product), fragment, aggregate or derivative of the desired polypeptide (e.g., high molecular weight species (HMWS) or very high molecular weight species (vHMWS) of the desired polypeptide); mis-paired bispecific variants; another polypeptide; endotoxin; viral contaminant; cell culture media component, etc. In some examples, the contaminant may be a host cell protein (HCP) from, for example but not limited to, a bacterial cell such as an E. coli cell, an insect cell, a prokaryotic cell, a eukaryotic cell, a yeast cell, a mammalian cell, an avian cell, a fungal cell.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

### Single Pass Tangential Flow Filtration

Traditional tangential flow filtration (TFF) operates in batch mode, where the feed/retentate is recirculated tangentially across a semi-permeable filter membrane over multiple passes to achieve the desired concentration of product (e.g., a polypeptide product). Single-pass Tangential Flow Filtration (SPTFF) is a mode of TFF where the feed solution passes through the membranes only once, to produce a permeate stream and a concentrated retentate stream. Increased residence time in the feed channel, achieved by reduced feed flow rate or increased path length in a serial configuration, can improve conversion of the feed solution to the desired product concentration.

As noted above, in some aspects, provided herein are methods for purifying a polypeptide (e.g., an antibody) from a polypeptide preparation, comprising the following steps in order: a) subjecting the polypeptide preparation to one or more purification processes and recovering a first resulting pool having a viscosity of 5-40 cP; b) subjecting the pool recovered from step a) to a SPTFF operation comprising use of membranes in one or both of a serial membrane configuration and/or a parallel membrane configuration and recovering a second resulting pool having an operating viscosity of 41-400 cP, wherein, i) the operating temperature is 15-55° C., ii) the feed flux is 5-50 L/m²/hr (LMH).

In some embodiments, the viscosity of the recovered pool in step a) is about any of 5-40 cP, 10-40 cP, 15-40 cP, 20-40 cP, 25-40 cP, 30-40 cP, 35-40 cP, 5-35 cP, 10-35 cP, 15-35 cP, 20-35 cP, 25-35 cP, 30-35 cP, 5-30 cP, 10-30 cP, 15-30 cP, 20-30 cP, 25-30 cP, 5-25 cP, 10-25 cP, 15-25 cP, 20-25 cP, 5-20 cP, 10-20 cP, 15-20 cP, 5-15 cP, 10-15 cP, or 5-10 cP. In some embodiments, the viscosity of the recovered pool in step (a) is less than about any of 5 cP, 10 cP, 15 cP, 20 cP, 25 cP, 30 cP, 35 cP or 40 cP. In some embodiments, the viscosity of recovered pool in step (a) is determined by the running temperature of the SPTFF operation.

In some embodiments, the membrane configuration is a serial configuration in step b). The serial membrane configuration can include any configuration capable of producing a resulting pool having an operating viscosity in the ranges described herein. For instance, the serial membrane configuration can be 1:1:1 in series, wherein 1:1:1 represents a three-stage membrane configuration in which the first stage contains a single membrane, the second stage contains a single membrane, and the third stage contains a single membrane. The serial membrane can also be any one of 2:1:1 in series, 2:2:1 in series, 2:1:2 in series, 1:2:2 in series, 2:2:2 in series, 1:1:1:1 in series, 2:1:1:1 in series, 1:2:1:1 in series, 1:1:2:1 in series, 1:1:1:2 in series, 2:2:1:1 in series, 2:1:2:1 in series, 2:1:1:2 in series, 1:2:2:1 in series, 1:2:1:2 in series, 1:1:2:2 in series, 2:2:2:1 in series, 2:1:2:2 in series, 2:2:1:2 in series, 3:2:1 in series, 4:3:2 in series, 1:1:1:1:1 in series, or 1:2:2:2 in series. In each of the exemplary configurations listed above, each number represents the number of membranes in each stage of a membrane configuration. In some embodiments, the membrane configuration is a parallel configuration in step b). The parallel membrane configurations can include any configuration capable of producing a resulting pool having an operating viscosity in the ranges described herein.

In some embodiments, the membranes in step b) have a nominal molecular weight limit of about 10 kD, about 30 kD or about 50 kD. In some embodiments, one or more of the respective membranes in step b) can have a different molecular weight than one or more of the other respective membranes (e.g., a 1:1:1 series be 10 kD:30 kD:30 kD). In some embodiments, the membranes in step b) are narrow channel or wide channel membranes. In some embodiments, the membranes in step b) comprise a C screen or a D screen. In some embodiments, the membranes in step b) can be regenerated cellulose or PES. In some embodiments, the membranes in step b) are PELLICON XL membranes or SARTOCON HYDROSART® ECO membranes. In some embodiments, the membranes in step b) can be Pall membranes, Millipore membranes (e.g., Millipore A screens, C screens, D screens), Sartorius membranes, Repligen membranes (e.g., Repligen L screens, E screens), Centramate membranes, Delta membranes, Hydrosart, ECO, or any other membrane suitable to the SPTFF systems described herein. In some embodiments, the polypeptide preparation in step b) is formulated in a viscosity-reducing excipient, such as arginine or histidine.

In some embodiments, the operating temperature of the SPTFF is between about 15° C. and about 55° C., about 20° C. and about 55° C., about 20° C. and about 50° C., about 20° C. and about 40° C., about 20° C. and about 30° C., about 30° C. and about 55° C., about 30° C. and about 50° C., about 30° C. and about 40° C., about 40° C. and about 55° C., about 40° C. and about 50° C., about 50° C. and about 55° C. In some embodiments, the operating temperature of the SPTFF is less than about any of 20° C., 25° C., 30° C., 35° C., 40° C., 45° C., 50° C., 55° C.

In some embodiments, the feed flux is between about 5 and about 50 liters/meter²/hour (LMH). In some embodiments, the feed flux of the SPTFF is between about 5 and about 150 LMH. In some embodiments, the feed flux of the SPTFF is between about any of 5 and 25 LMH, 25 and 50 LMH, 50 and 75 LMH, 75 and 100 LMH, 100 and 125 LMH, 125 and 150 LMH, 5 and 50 LMH, 5 and 45 LMH, 5 and 40 LMH, 5 and 35 LMH, 5 and 30 LMH, 5 and 25 LMH, 5 and 20 LMH, 5 and 15 LMH, 5 and 10 LMH, 10 and 50 LMH, 10 and 45 LMH, 10 and 40 LMH, 10 and 35 LMH, 10 and 30 LMH, 10 and 25 LMH, 10 and 20 LMH, 10 and 15 LMH, 15 and 50 LMH, 15 and 45 LMH, 15 and 40 LMH, 15 and 35 LMH, 15 and 30 LMH, 15 and 25 LMH, 15 and 20 LMH, 20 and 50 LMH, 20 and 45 LMH, 20 and 40 LMH, 20 and 35 LMH, 20 and 30 LMH, 20 and 25 LMH, 25 and 50 LMH, 25 and 45 LMH, 25 and 40 LMH, 25 and 35 LMH, 25 and 30 LMH, 30 and 50 LMH, 30 and 45 LMH, 35 and 40 LMH, 40 and 50 LMH, 10 and 45 LMH, or 45 and 50 LMH). In some embodiments, the feed flux of the SPTFF is less than about any of 5 LMH, 10 LMH, 15 LMH, 20 LMH, 25 LMH, 30 LMH, 35 LMH, 40 LMH, 45 LMH, 50 LMH, 75 LMH, 100 LMH, 125 LMH, or 150 LMH.

In some embodiments, the operating viscosity of the SPTFF is between about 41 cP and about 400 cP. In some embodiments, the viscosity of the recovered pool in step b) is about any of 50-400 cP, 75-100 cP, 100-400 cP, 125-400 cP, 150-400 cP, 200-400 cP, 250-400 cP, 300-400 cP, 350-400 cP, 100-350 cP, 150-350 cP, 200-350 cP, 250-350 cP, 300-350 cP, 100-300 cP, 150-300 cP, 200-300 cP, 250-300 cP, 100-250 cP, 150-250 cP, 200-250 cP, 100-200 cP, 150-200 cP, or 100-150 cP. In some embodiments, the viscosity of the recovered pool in step (a) is less than about any of 50 cP, 100 cP, 150 cP, 200 cP, 250 cP, 300 cP, 350 cP or 400 cP. In some embodiments, the viscosity of recovered pool in step (a) is determined at the operating temperature of the SPTFF operation.

In some embodiments, the retentate pressure of the SPTFF operation is between about 5 psi and about 20 psi. In some embodiments, the retentate pressure of the SPTFF operation is between about 5 psi and about 15 psi. In some embodiments, the retentate pressure of the SPTFF operation is between about 5 psi and about 20 psi, about 5 psi and about 15 psi, about 5 psi and about 10 psi, or about 10 psi and about 15 psi.

In some embodiments, the feed pressure of the SPTFF operation is between about 5 psi and about 50 psi. In some embodiments, the feed pressure of the SPTFF operation is between about 5 psi and about 40 psi, 5 psi and about 35 psi, about 5 psi and about 30 psi, about 5 psi and about 25 psi, about 5 psi and about 20 psi, about 5 psi and about 15 psi, about 5 psi and about 10 psi, about 10 psi and about 50 psi, about 10 psi and about 40 psi, 10 psi and about 35 psi, about 10 psi and about 30 psi, about 10 psi and about 25 psi, about 10 psi and about 20 psi, about 10 psi and about 15 psi, 15 psi and about 50 psi, about 15 psi and about 40 psi, 15 psi and about 35 psi, about 15 psi and about 30 psi, about 15 psi and about 25 psi, about 15 psi and about 20 psi, 20 psi and about 50 psi, about 20 psi and about 40 psi, 20 psi and about 35 psi, about 20 psi and about 30 psi, about 20 psi and about 25 psi, 25 psi and about 50 psi, about 25 psi and about 40 psi, 25 psi and about 35 psi, about 25 psi and about 30 psi, 30 psi and about 50 psi, about 30 psi and about 40 psi, 30 psi and about 35 psi, 35 psi and about 50 psi, about 35 psi and about 40 psi, or 40 psi and about 50 psi.

In some embodiments, the transmembrane pressure (TMP) of the SPTFF operation is between about 5 psid and about 15 psid. In some embodiments, the TMP of the SPTFF operation is between about 5 psid and about 25 psid. In some embodiments, the TMP of the SPTFF operation is between about 5 psid and about 20 psid, about 5 psid and about 15 psid, about 5 psi and about 10 psid, or about 10 psid and about 25 psid.

In some embodiments, the SPTFF operation achieves a volume concentration factor (VCF) of between about 1.2 and about 2.5. In some embodiments, the SPTFF operation achieves a VCF of between about any of 1.2 and 2.5, 1.5 and 2.5, 1.6 and 2.5, 1.7 and 2.5, 1.8 and 2.5, 1.9 and 2.5, 2.0 and 2.5, 2.1 and 2.5, 2.2 and 2.5, 2.3 and 2.5, 2.4 and 2.5, 1.2 and 2.4, 1.5 and 2.4, 1.6 and 2.4, 1.7 and 2.4, 1.8 and 2.4, 1.9 and 2.4, 2.0 and 2.4, 2.1 and 2.4, 2.2 and 2.4, 2.3 and 2.4, 1.2 and 2.3, 1.5 and 2.3, 1.6 and 2.3, 1.7 and 2.3, 1.8 and 2.3, 1.9 and 2.3, 2.0 and 2.3, 2.1 and 2.3, 2.2 and 2.3, 1.2 and 2.2, 1.5 and 2.52 1.6 and 2.2, 1.7 and 2.2, 1.8 and 2.2, 1.9 and 2.2, 2.0 and 2.2, 2.1 and 2.2, 1.2 and 2.1, 1.5 and 2.1, 1.6 and 2.1, 1.7 and 2.1, 1.8 and 2.1, 1.9 and 2.1, 2.0 and 2.1, 1.2 and 2.0, 1.5 and 2.0, 1.6 and 2.0, 1.7 and 2.0, 1.8 and 2.0, 1.9 and 2.0, 1.2 and 1.9, 1.5 and 1.9, 1.6 and 1.9, 1.7 and 1.9, 1.8 and 1.9, 1.2 and 1.8, 1.5 and 1.8, 1.6 and 1.8, 1.7 and 1.8, 1.2 and 1.7, 1.5 and 1.7, 1.6 and 1.7, 1.2 and 1.6, 1.5 and 1.6, or 1.2 and 1.5. In some embodiments, the SPTFF operation achieves a VCF of any of about 1.2, 1.3, 1.4, 1.5, 1.6, 1.7, 1.8, 1.9, 2.0, 2.1, 2.2, 2.3, 2.4, 2.5 or greater than 2.5.

In some embodiments, the retentate concentration following the SPTFF operation is between about 100 g/L and 350 g/L. In some embodiments, the retentate concentration following the SPTFF operation is between about any of 100 g/L and 350 g/L, 125 g/L and 350 g/L, 150 g/L and 350 g/L, 175 g/L and 350 g/L, 200 g/L and 350 g/L, 225 g/L and 350 g/L, 250 g/L and 350 g/L, 275 g/L and 350 g/L, 300 g/L and 350 g/L, 325 g/L and 350 g/L, 100 g/L and 325 g/L, 125 g/L and 325 g/L, 150 g/L and 325 g/L, 175 g/L and 325 g/L, 200 g/L and 325 g/L, 225 g/L and 325 g/L, 250 g/L and 3525 g/L, 275 g/L and 325 g/L, 300 g/L and 325 g/L, 100 g/L and 300 g/L, 125 g/L and 300 g/L, 150 g/L and 300 g/L, 175 g/L and 300 g/L, 200 g/L and 300 g/L, 225 g/L and 300 g/L, 250 g/L and 300 g/L, 275 g/L and 300 g/L, 100 g/L and 275 g/L, 125 g/L and 275 g/L, 150 g/L and 275 g/L, 175 g/L and 275 g/L, 200 g/L and 275 g/L, 225 g/L and 275 g/L, 250 g/L and 275 g/L, 100 g/L and 250 g/L, 125 g/L and 250 g/L, 150 g/L and 250 g/L, 175 g/L and 250 g/L, 200 g/L and 250 g/L, 225 g/L and 250 g/L, 100 g/L and 225 g/L, 125 g/L and 225 g/L, 150 g/L and 225 g/L, 175 g/L and 225 g/L, 200 g/L and 225 g/L, 100 g/L and 200 g/L, 125 g/L and 200 g/L, 150 g/L and 200 g/L, 175 g/L and 200 g/L, 100 g/L and 175 g/L, 125 g/L and 175 g/L, 150 g/L and 175 g/L, 100 g/L and 150 g/L, 125 g/L and 150 g/L, or 100 g/L and 125 g/L. In some embodiments, the retentate concentration following the SPTFF operation is more than about 100 g/L, 125 g/L, 150 g/L, 175 g/L, 200 g/L, 250 g/L, 275 g/L, 300 g/L, 325 g/L, or 350 g/L.

In some embodiments of the purification processes, the operating temperature is between about 30° C. and about 55° C., the feed flux is between about 5 LMH and about 50 LMH, and the feed concentration is about 100 to about 200 g/L. In some embodiments of the purification processes, the operating temperature is between about 30° C. and about 55° C., the feed flux is between about 25 LMH and about 50 LMH, the feed concentration is about 100 to about 150 g/L, and the SPTFF membranes are configured in a 1:1:1 or 1:1:1:1 in series. In some embodiments of the purification processes, the operating temperature is between about 40° C. and about 55° C., the feed flux is between about 10 LMH and about 15 LMH, and the feed concentration is about 140 to about 170 g/L. In some embodiments of the purification processes, the operating temperature is between about 40° C. and about 55° C., the feed flux is between about 10 LMH and about 15 LMH, the feed concentration is about 140 to about 170 g/L, and the SPTFF membranes are configured in a 1:1:1 or 1:1:1:1 in series. In some embodiments of the purification processes, the operating temperature is between about 30° C. and about 55° C., the feed flux is between about 5 LMH and about 50 LMH, the TMP is between about 5 psid and about 15 psid, and the feed concentration is about 100 to about 200 g/L. In some embodiments of the purification processes, the operating temperature is between about 30° C. and about 55° C., the feed flux is between about 25 LMH and about 50 LMH, the TMP is between about 5 psid and about 15 psid, the feed concentration is about 100 to about 150 g/L, and the SPTFF membranes are configured in a 1:1:1 or 1:1:1:1 in series. In some embodiments of the purification processes, the operating temperature is between about 40° C. and about 55° C., the feed flux is between about 10 LMH and about 15 LMH, the TMP is between about 5 psid and about 10 psid, and the feed concentration is about 140 to about 170 g/L. In some embodiments of the purification processes, the operating temperature is between about 40° C. and about 55° C., the feed flux is between about 10 LMH and about 15 LMH, the TMP is between about 5 psid and about 10 psid, the feed concentration is about 140 to about 170 g/L, and the SPTFF membranes are configured in a 1:1:1 or 1:1:1:1 in series.

In some embodiments of the purification processes, the operating temperature is between about 30° C. and about 50° C., the feed flux is between about 5 LMH and about 50 LMH, and the feed concentration is about 100 to about 200 g/L. In some embodiments of the purification processes, the operating temperature is between about 30° C. and about 50° C., the feed flux is between about 25 LMH and about 50 LMH, the feed concentration is about 100 to about 150 g/L, and the SPTFF membranes are configured in a 1:1:1 or 1:1:1:1 in series. In some embodiments of the purification processes, the operating temperature is between about 40° C. and about 50° C., the feed flux is between about 10 LMH and about 15 LMH, and the feed concentration is about 140 to about 170 g/L. In some embodiments of the purification processes, the operating temperature is between about 40° C. and about 50° C., the feed flux is between about 10 LMH and about 15 LMH, the feed concentration is about 140 to about 170 g/L, and the SPTFF membranes are configured in a 1:1:1 or 1:1:1:1 in series. In some embodiments of the purification processes, the operating temperature is between about 30° C. and about 50° C., the feed flux is between about 5 LMH and about 50 LMH, the TMP is between about 5 psid and about 15 psid, and the feed concentration is about 100 to about 200 g/L. In some embodiments of the purification processes, the operating temperature is between about 30° C. and about 50° C., the feed flux is between about 25 LMH and about 50 LMH, the TMP is between about 5 psid and about 15 psid, the feed concentration is about 100 to about 150 g/L, and the SPTFF membranes are configured in a 1:1:1 or 1:1:1:1 in series. In some embodiments of the purification processes, the operating temperature is between about 40° C. and about 50° C., the feed flux is between about 10 LMH and about 15 LMH, the TMP is between about 5 psid and about 10 psid, and the feed concentration is about 140 to about 170 g/L. In some embodiments of the purification processes, the operating temperature is between about 40° C. and about 50° C., the feed flux is between about 10 LMH and about 15 LMH, the TMP is between about 5 psid and about 10 psid, the feed concentration is about 140 to about 170 g/L, and the SPTFF membranes are configured in a 1:1:1 or 1:1:1:1 in series.

In some embodiments of the invention, the one or more purification processes of step a) comprises an ultrafiltration operation. In some embodiments, the one or more purification processes of step a) comprises at least two ultrafiltration operations. In some embodiments, the one or more purification processes of step a) comprises a diafiltration. In some embodiments, step a) comprises i. subjecting the polypeptide preparation to an ultrafiltration operation to a first target concentration, ii. subjecting the polypeptide preparation to a diafiltration operation for buffer exchange, and iii. subjecting the polypeptide preparation to a second ultrafiltration operation to a second target concentration and recovering the resulting pool having a viscosity of 5-40 cP.

In some aspects, the invention provides methods for purifying a polypeptide (e.g., an antibody) from a polypeptide preparation, comprising the following steps in order: a) i) subjecting the polypeptide preparation to an ultrafiltration operation to a first target concentration, ii) subjecting the polypeptide preparation to a diafiltration operation for buffer exchange, iii) subjecting the polypeptide preparation to a second ultrafiltration operation to a second target concentration and recovering the resulting pool having a viscosity of 5-40 cP; and b) subject the pool recovered from step a) to a SPTFF operation comprising use of membranes in either or both of a serial membrane configuration and/or a parallel membrane configuration then recover the resulting pool having an operating viscosity of 41-400 cP, wherein, i) the operating temperature is 15-55° C., ii) the feed flux is 5-50 L/m²/hr (LMH).

In some embodiments, the first target concentration of the ultrafiltration operation is between about 5 g/L and 80 g/L. In some embodiments, the first target concentration of the ultrafiltration operation is between about any of 5 g/L and 80 g/L, 10 g/L and 80 g/L, 20 g/L and 80 g/L, 30 g/L and 80 g/L, 40 g/L and 80 g/L, 50 g/L and 80 g/L, 60 g/L and 80 g/L, 70 g/L and 80 g/L, 5 g/L and 70 g/L, 10 g/L and 70 g/L, 20 g/L and 70 g/L, 30 g/L and 70 g/L, 40 g/L and 70 g/L, 50 g/L and 70 g/L, 60 g/L and 70 g/L, 5 g/L and 60 g/L, 10 g/L and 60 g/L, 20 g/L and 60 g/L, 30 g/L and 60 g/L, 40 g/L and 60 g/L, 50 g/L and 60 g/L, 5 g/L and 50 g/L, 10 g/L and 50 g/L, 20 g/L and 50 g/L, 30 g/L and 50 g/L, 40 g/L and 50 g/L, 5 g/L and 40 g/L, 10 g/L and 40 g/L, 20 g/L and 40 g/L, 30 g/L and 40 g/L, 5 g/L and 30 g/L, 10 g/L and 30 g/L, 20 g/L and 30 g/L, 5 g/L and 20 g/L, 10 g/L and 20 g/L, or 5 g/L and 10 g/L. In some embodiments, the first target concentration of the ultrafiltration operation is any of about 5 g/L, 10 g/L, 15 g/L, 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, or more than 80 g/L.

In some embodiments, the buffer exchange of the diafiltration is about a 6-12× exchange. In some embodiments, the buffer exchange of the diafiltration is any of about a 5-15×, 5-12×, 5-10×, 5-9×, 5-8×, 5-7×, 5-6×, 6-15×, 6-12×, 6-10×, 6-9×, 6-8×, 6-7×, 7-15×, 7-12×, 7-10×, 7-9×, 7-8×, 8-15×, 8-12×, 8-10×, 8-9×, 9-15×, 9-12×, 9-10×, 10-15×, 10-12×, or 12-15× exchange. In some embodiments, the buffer exchange of the diafiltration is any of about a 5×, 6×, 7×, 8×, 9×, 10×, 11×, 12×, 13×, 14×, 15×, or greater than 15× exchange.

In some embodiments, the second target concentration of the ultrafiltration operation is between about 100 g/L and 200 g/L. In some embodiments, the second target concentration of the ultrafiltration operation is between about any of 25 g/L and 250 g/L, 50 g/L and 250 g/L, 75 g/L and 250 g/L, 100 g/L and 250 g/L, 125 g/L and 250 g/L, 150 g/L and 250 g/L, 175 g/L and 250 g/L, 200 g/L and 250 g/L, 225 g/L and 250 g/L, 25 g/L and 225 g/L, 50 g/L and 225 g/L, 75 g/L and 225 g/L, 100 g/L and 225 g/L, 125 g/L and 225 g/L, 150 g/L and 225 g/L, 175 g/L and 225 g/L, 200 g/L and 225 g/L, 25 g/L and 200 g/L, 50 g/L and 200 g/L, 75 g/L and 200 g/L, 100 g/L and 200 g/L, 125 g/L and 200 g/L, 150 g/L and 200 g/L, 175 g/L and 200 g/L, 25 g/L and 175 g/L, 50 g/L and 175 g/L, 75 g/L and 175 g/L, 100 g/L and 175 g/L, 125 g/L and 175 g/L, 150 g/L and 175 g/L, 25 g/L and 150 g/L, 50 g/L and 150 g/L, 75 g/L and 150 g/L, 100 g/L and 150 g/L, 125 g/L and 150 g/L, 25 g/L and 125 g/L, 50 g/L and 125 g/L, 75 g/L and 125 g/L, 100 g/L and 125 g/L, 25 g/L and 100 g/L, 50 g/L and 100 g/L, 75 g/L and 100 g/L, 25 g/L and 75 g/L, 50 g/L and 75 g/L, or 25 g/L and 50 g/L. In some embodiments, the second target concentration of the ultrafiltration operation is any of about 25 g/L, 50 g/L, 75 g/L, 100 g/L, 125 g/L, 150 g/L, 175 g/L, 200 g/L, 225 g/L, 250 g/L, or more than 250 g/L.

In some embodiments, the purification processes of step a) are operated in-line with the SPTFF operation of step b). In some embodiments, the ultrafiltration processes of step a are operated in-line with the SPTFF operation of step b. In some embodiments, the ultrafiltration/diafiltration/ultrafiltration processes of step a are operated in-line with the SPTFF operation of step b. In some embodiments, the purification processes of step a) and the SPTFF operation of step b) are performed as discrete steps (i.e., the purification processes of step a) are not operated in-line with the SPTFF operation of step b).

### Conditioning Steps

In some embodiments, the purification processes described with reference to step a) comprise a conditioning step. In some embodiments, the conditioning step is performed after a capture step. The capture step may include an ultrafiltration/diafiltration (UF/DF) step. Conditioning steps, including what is involved with processing for a conditioning step, are known in the art. See, e.g., Liu et al. mAbs, 2, 2010, which is hereby incorporated by reference.

In some embodiments, the conditioning step comprises a virus inactivation step, such as a low pH hold step. In some embodiments, the low pH hold step is performed at a pH of about 2.5 to about 4. In some embodiments, the low pH hold step is configured for viral inactivation. In some embodiments, the low pH hold step is capable of inactivating endogenous/adventitious viruses.

In some embodiments, viscosity reducing excipients such as arginine and histidine, or other excipients containing hydrophobic moieties, can be controlled by a diafiltration operation prior to the SPTFF operation. Changing solution pH may also affect the viscosity.

### Virus Filtration Steps

In some embodiments, the purification processes described with reference to step a) comprise a virus filtration step. In some embodiments, the virus filtration step is performed after one or more purification steps.

Virus filtration steps, including what is involved with processing for a virus filtration step, are known in the art. See, e.g., Liu et al. mAbs, 2, 2010, and U.S. Application No. 20140309403, which are hereby incorporated by reference.

In some embodiments, the virus filtration step comprises processing via a virus filter. In some embodiments, the virus filtration step comprises a pH hold step. In some embodiments, processing via a virus filter is performed after the pH hold step.

### Alternative Purification Steps

In some embodiments, the purification processes of step a) are chromatography processes. In some embodiments, the chromatography processes are column chromatography processes. In some embodiments, the chromatography processes are one or more of an affinity chromatography, an ion exchange chromatography, and a mixed mode chromatography. In some embodiments, the chromatography processes include hydrophobic interaction chromatography (HIC). In some embodiments, the chromatography processes comprise an affinity chromatography, an ion exchange chromatography and a mixed mode chromatography. In some embodiments, the chromatography processes comprise, in order, an affinity chromatography, an ion exchange chromatography and a mixed mode chromatography. In some embodiments, the chromatography processes comprise an affinity chromatography, a cation ion exchange chromatography and an anion exchange mixed mode chromatography. In some embodiments, the chromatography processes comprise, in order, an affinity chromatography, a cation exchange chromatography and an anion exchange mixed mode chromatography. In some embodiments, the affinity chromatography is used in bind-and-elute mode and/or the cation exchange chromatography is used in overload mode, and/or the anion exchange mixed mode chromatography is used in flow-through mode. In some embodiments, one or more of the affinity chromatography, cation exchange chromatography, and anion exchange mixed mode chromatography can be used in any other suitable modes. For instance, the cation exchange chromatography can be used in flow-through mode and the anion exchange mixed mode chromatography can be used in overload mode. It should be understood that the aforementioned chromatography configurations are meant to be exemplary, and any suitable configurations may be implemented without deviating from the scope of the claims. In some embodiments, the SPTFF is used in-line with the one or more chromatography processes.

In some embodiments, the purification process comprises an affinity chromatography. In some embodiments, the affinity chromatography is protein A chromatography. Examples of affinity chromatography include, but are not limited to chromatography using derivatized with protein A, protein G, or protein L. Examples of affinity chromatography material include, but are not limited to, ProSep®-vA, ProSep® Ultra Plus, Protein A Sepharose® Fast Flow, Toyopearl® AF-rProtein A, MabSelect™, MabSelect SuRe™ and MabSelect SuRe™ LX, MabSelect PrismA™, Capto™ L, MabSelect™ VL, and Praesto® A50+. It should be understood that the aforementioned list of affinity chromatography material is meant to be exemplary. Any suitable affinity chromatography material may be used without deviating from the scope of the claims. In some embodiments of the above, the affinity chromatography material is in a column. In some embodiments of the above, the affinity chromatography material is a membrane. In some embodiments, the affinity chromatography is protein A chromatography. In some embodiments, the protein A chromatography is MabSelect SuRe™ chromatography.

In some embodiments, the purification process comprises a cation exchange chromatography. In some embodiments, the cation exchange chromatography material is a solid phase that is positively charged and has free cations for exchange with cations in an aqueous solution passed over or through the solid phase. In some embodiments of any of the methods described herein, the cation exchange material may be a membrane, a monolith, or resin. In some embodiments, the cation exchange material may be a resin. The cation exchange material may comprise a carboxylic acid functional group or a sulfonic acid functional group such as, but not limited to, sulfonate, carboxylic, carboxymethyl sulfonic acid, sulfoisobutyl, sulfoethyl, carboxyl, sulphopropyl, sulphonyl, sulphoxyethyl, or orthophosphate. In some embodiments of the above, the cation exchange chromatography material is a cation exchange chromatography column. In some embodiments of the above, the cation exchange chromatography material is a cation exchange chromatography membrane. Examples of cation exchange materials are known in the art include, but are not limited to Mustang®S, Sartobind® S, SO3 Monolith, CIM®, CIMmultus® and CIMac® SO3, S Ceramic HyperD®, POROS® XS, POROS® HS 50, POROS® HS 20, sulphopropyl-Sepharose® Fast Flow (SPSFF), SP-Sepharose® XL (SPXL), CM Sepharose® Fast Flow, Capto™ S, Fractogel® EMD Se Hicap, Fractogel® EMD SO3-, or Fractogel® EMD COO—, or Eshmuno® S resin. In some embodiments of any of the methods described herein, the cation exchange material is POROS® HS 50. In some embodiments, the cation exchange chromatography is performed in bind and elute mode. In some embodiments of the above, the cation exchange chromatography material is in a column. In some embodiments of the above, the cation exchange chromatography material is in a membrane.

In some aspects, the polypeptide preparation or the eluate from the affinity chromatography is loaded onto an ion exchange chromatography material at an amount exceeding the dynamic binding capacity of the chromatography material for the polypeptide (e.g., an antibody). In the process of loading the ion exchange chromatography material, some of the polypeptide will break-through in the wash and some of the polypeptide will remain bound to the chromatography material. The dynamic binding capacity of a chromatography material for a polypeptide and for one or more impurities can be estimated by determining the partition coefficient (Kₚ) for the product or impurities as a function of pH and counterion concentration for a particular chromatography material. For example, the dynamic binding capacity of a chromatography material, e.g. a cation exchange resin, for a polypeptide may be determined. Actual binding capacities of a chromatography material for a product or contaminant at a specific combination of pH and counterion concentration can be determined by challenging the binding with an excess of the polypeptide and/or impurities. In some embodiments, the ion exchange chromatography is performed where the Kₚ of the product, e.g. polypeptide, is greater than about 30. In some embodiments, the ion exchange chromatography is performed where the Kₚ of the product is greater than about 50. In some embodiments, the ion exchange chromatography is performed where the Kₚ of the product is greater than about 75. In some embodiments, the ion exchange chromatography is performed where the Kₚ of the product is greater than about 100. In some embodiments, the ion exchange chromatography is a cation exchange chromatography.

In some embodiments of any of the methods described herein, the polypeptide preparation or the eluate from the affinity chromatograph is loaded onto an ion exchange chromatography material (e.g., a cation exchange chromatography material) at a loading density of the polypeptide of greater than about any of 30 g/L, 40 g/L, 50 g/L, 60 g/L, 70 g/L, 80 g/L, 90 g/L, 100 g/L, 110 g/L, 120 g/L, 130 g/L, 140 g/L, 150 g/L, 160 g/L, 170 g/L, 180 g/L, 190 g/L, 200 g/L, 300 g/L, 400 g/L, 500 g/L, 550 g/L, 600 g/L, 650 g/L, 700 g/L, 800 g/L, 900 g/L, 1000 g/L, 2000 g/L or 5000 g/L of the ion exchange chromatography material. In some embodiments, the polypeptide preparation or the eluate from the affinity chromatograph is loaded onto an ion exchange chromatography material (e.g., a cation exchange chromatography material) at a loading density of less than about any of 30 g/L, 40 g/L, 50 g/L, 60 g/L, 70 g/L, 80 g/L, 90 g/L, 100 g/L, 110 g/L, 120 g/L, 130 g/L, 140 g/L, 150 g/L, 160 g/L, 170 g/L, 180 g/L, 190 g/L, 200 g/L, 300 g/L, 400 g/L, 500 g/L, 550 g/L, 600 g/L, 650 g/L, 700 g/L, 800 g/L, 900 g/L, 1000 g/L or 2000 g/L of the ion exchange chromatography material. The polypeptide preparation or the eluate from the affinity chromatograph may be loaded onto an ion exchange chromatography material (e.g., a cation exchange chromatography material) at a loading density of the polypeptide of between about any of 30 g/L and 2000 g/L, 30 g/L and 1000 g/L, 30 g/L and 200 g/L, 30 g/L and 180 g/L, 50 g/L and 2000 g/L, 50 g/L and 1000 g/L, 50 g/L and 200 g/L, 50 g/L and 180 g/L, 150 g/L and 2000 g/L, 150 g/L and 1500 g/L, 150 g/L and 1000 g/L, 200 g/L and 1000 g/L, 200 g/L and 1500 g/L, 300 g/L and 1500 g/L, 400 g/L and 1000 g/L, or 500 g/L and 1000 g/L of the ion exchange chromatography material.

In some embodiments, the purification process comprises a mixed mode chromatography. In some embodiments, the mixed-mode material comprises functional groups capable of one of more of the following functionalities: anionic exchange, cation exchange, hydrogen bonding, and hydrophobic interactions. In some embodiments, the mixed-mode material comprises functional groups capable of anionic exchange and hydrophobic interactions. In some embodiments, the mixed-mode material comprises functional groups capable of cationic exchange and hydrophobic interactions. The mixed-mode material may contain N-benzyl-N-methyl ethanol amine, 4-mercapto-ethyl-pyridine, hexylamine, or phenylpropylamine as ligand or contain cross-linked polyallylamine. Examples of the mixed-mode materials include Capto™ adhere resin, Accell™ Plus Quaternary Methyl Amine (QMA) resin, Capto™ MMC resin, MEP HyperCel™ resin, HEA HyperCel™ resin, PPA HyperCel™ resin, or ChromaSorb™ membrane or Sartobind STIC®, or Eschmuno® CMX, or Eschmuno® HCX. In some embodiments, the mixed-mode material is Capto™ adhere resin. In some embodiments, the mixed-mode chromatography is performed in flow-through mode where the antibody flows through the mixed-mode chromatography material. In some embodiments of the above, the mixed-mode material is in a column. In some embodiments of the above, the mixed-mode material is in a membrane.

In some aspects, the present disclosure provides additional steps involved or associated with a purification and SPTFF processes described herein. Additional steps involved or associated with a purification and SPTFF processes, and methods for conducting such steps, are known. See, e.g., Liu et al., mAbs, 2, 2010, which is hereby incorporated by reference in its entirety.

In some embodiments, the purification and SPTFF processes further comprise a sample processing step, such as a sample preparation step. In some embodiments, the preparatory step comprises sample conditioning (e.g., buffer additions/exchange, dilution, pH adjustment). In some embodiments, the purification and SPTFF processes further comprises a clarification step, such as to clarify HCCF. In some embodiments, the purification and SPTFF processes further comprises a host cell and host cell debris removal step, such as to remove host cells and host cell debris from a sample and/or a composition obtained from the purification and SPTFF processes. In some embodiments, the purification and SPTFF processes further comprises a centrifugation step. In some embodiments, the purification and SPTFF processes further comprises a sterile and/or guard filtration step. In some embodiments, the purification and SPTFF processes further comprises a tangential flow micro-filtration step. In some embodiments, the purification and SPTFF processes further comprises a Tangential Flow Depth Filtration (TFDF) step. In some embodiments, the purification and SPTFF processes further comprises a flocculation/precipitation step. In some embodiments, the purification and SPTFF processes further comprises a depth filtration step.

Samples, Components Thereof, and Compositions Obtained from Purification Processes

In some aspects, the SPTFF described herein are useful for purifying and concentrating a polypeptide from a polypeptide preparation. In some embodiments, the polypeptide preparation is derived from a host cell preparation. In some embodiments, the host cell preparation is a host cell culture fluid (HCCF). In some embodiments, the polypeptide preparation comprises a portion of a host cell culture fluid. In some embodiments, the polypeptide preparation is derived from a host cell culture fluid. In some embodiments, the polypeptide preparation comprises a host cell. In some embodiments, the polypeptide preparation comprises a component of a host cell, such as host cell debris. In some embodiments, the host cell is a bacterial cell. In some embodiments, the host cell is an insect cell. In some embodiments, the host cell is a mammalian cell. In some embodiments, the host cell is a Chinese hamster ovary (CHO) cell. In some embodiments, the host cell is an E. coli cell.

The methods described herein are used in the purification of a polypeptide from a polypeptide preparation. In some embodiments, the polypeptide is an antibody. In some embodiments, the antibody is a monoclonal antibody. In some embodiments, the antibody is a humanized antibody. In some embodiments, the antibody is selected from the group consisting of an anti-CD20 antibody, an anti-CD40 antibody, an anti-HER2 antibody, an anti-IL6 antibody, an anti-IgE antibody, an anti-IL13 antibody, an anti-TIGIT antibody, an anti-PD-L1 antibody, an anti-VEGF-A antibody, an anti-VEGF-A/ANG2 antibody, an anti-CD79b antibody, an anti-ST2 antibody, an anti-factor D antibody, an anti-factor IX antibody, an anti-factor X antibody, an anti-abeta antibody, an anti-tau antibody, an anti-CEA antibody, an anti-CEA/CD3 antibody, an anti-CD20/CD3 antibody, an anti-FcRH5/CD3 antibody, an anti-Her2/CD3 antibody, an anti-FGFR1/KLB antibody, a FAP-4-1 BBL fusion protein, and a FAP-IL2v fusion protein. In some embodiments, the antibody is selected from the group consisting of ocrelizumab, pertuzumab, trastuzumab, tocilizumab, faricimab, polatuzumab, gantenerumab, cibisatamab, crenezumab, mosunetuzumab, tiragolumab, bevacizumab, rituximab, atezolizumab, obinutuzumab, lampalizumab, lebrikizumab, omalizumab ranibizumab, emicizumab, selicrelumab, prasinezumab, RO6874281, and RO7122290. In some embodiments, the antibody is an antigen binding fragment. In some embodiments, the antigen binding fragment is a Fab fragment. In some embodiments, the polypeptide is a Fab-ha conjugate.

In some embodiments, the sample comprises one or more host cell proteins. In some embodiments, the host cell protein is a hydrolytic enzyme. In some embodiments, the hydrolytic enzyme is a lipase, an esterase, a thioesterase, a phospholipase, or a ceramidase. In some embodiments, the hydrolytic enzyme is a multi-enzyme protein. In some embodiments, the multi-enzyme protein is a fatty acid synthase. In some embodiments, the fatty acid synthase comprises a thioesterase subunit.

The purification and SPTFF processes described herein, in some instances, may comprise numerous purification steps. In some embodiments, the term “composition” is used herein to describe any input (except the initial sample input to the purification process prior to the SPTFF processes), or output (intermediate or final) of any stage of the purification and SPTFF processes. For example, in some embodiments, use of the term “composition” is not limited to describing the final output of the purification and SPTFF processes.

### Additional Steps

In some aspects, the present disclosure provides additional steps involved or associated with a purification platform described herein. Additional steps involved or associated with a purification platform, and methods for conducting such steps, are known. See, e.g., Liu et al., mAbs, 2, 2010, which is hereby incorporated by reference in its entirety.

In some embodiments, the purification platform further comprises a sample processing step, such as a sample preparation step. In some embodiments, the purification platform further comprises a clarification step, such as to clarify HCCF. In some embodiments, the purification platform further comprises a host cell and host cell debris removal step, such as to remove host cells and host cell debris from a sample and/or a composition obtained from the purification platform. In some embodiments, the purification platform further comprises a centrifugation step. In some embodiments, the purification platform further comprises a sterile filtration step. In some embodiments, the purification platform further comprises a tangential flow micro-filtration step. In some embodiments, the purification platform further comprises a flocculation/precipitation step. In some embodiments, the purification platform further comprises a depth filtration step. In some embodiments, the purification platform further comprises a TFDF step.

### Pharmaceutical Compositions

In some aspects, the present disclosure provides pharmaceutical compositions obtained from the purification and SPTFF processes described herein. In some embodiments, the pharmaceutical composition is obtained from a method described herein. In some embodiments, the pharmaceutical composition is a purified composition. In some embodiments, the pharmaceutical composition is a sterile pharmaceutical composition.

In some embodiments, the antibody is a monoclonal antibody. In some embodiments, the antibody is a human, humanized, or chimeric antibody. In some embodiments, the antibody is selected from the group consisting of an anti-CD20 antibody, an anti-CD40 antibody, an anti-HER2 antibody, an anti-IL6 antibody, an anti-IgE antibody, an anti-IL13 antibody, an anti-TIGIT antibody, an anti-PD-L1 antibody, an anti-VEGF-A antibody, an anti-VEGF-A/ANG2 antibody, an anti-CD79b antibody, an anti-ST2 antibody, an anti-factor D antibody, an anti-factor IX antibody, an anti-factor X antibody, an anti-abeta antibody, an anti-tau antibody, an anti-CEA antibody, an anti-CEA/CD3 antibody, an anti-CD20/CD3 antibody, an anti-FcRH5/CD3 antibody, an anti-Her2/CD3 antibody, an anti-FGFR1/KLB antibody, a FAP-4-1 BBL fusion protein, and a FAP-IL2v fusion protein.

In some embodiments, the antibody is selected from the group consisting of ocrelizumab, pertuzumab, trastuzumab, tocilizumab, faricimab, polatuzumab, gantenerumab, cibisatamab, crenezumab, mosunetuzumab, tiragolumab, bevacizumab, rituximab, atezolizumab, obinutuzumab, lampalizumab, lebrikizumab, omalizumab ranibizumab, emicizumab, selicrelumab, prasinezumab, RO6874281, and RO7122290.

### Exemplary Embodiments

1. A method for purifying a polypeptide from a polypeptide preparation, comprising the following steps in order:
a) subjecting the polypeptide preparation to one or more purification processes and recovering a first resulting pool having a viscosity of about 5-40 cP; and
b) subjecting the pool recovered from step a) to a single pass tangential flow filtration (SPTFF) operation and recovering a second resulting pool having an operating viscosity of about 41-400 cP,
wherein,
   i) the operating temperature is about 15-55° C., and
   ii) the feed flux is about 5-50 L/m²/hr (LMH).
      2. The method of embodiment 1, wherein in step a) the viscosity of the first recovered pool is 10-35 cP.
      3. The method of embodiment 1 or 2, wherein in step b) the STPFF operation comprises use of a serial membrane configuration.
      4. The method of any one of embodiments 1-3, wherein in step b) the STPFF operation comprises use of a parallel membrane configuration.
      5. The method of any one of embodiments 3-4, wherein in step b) the SPTFF operation comprises use of both a serial membrane configuration and a parallel membrane configuration.
      6. The method of any one of embodiments 3-5, wherein in step b) wherein the membrane configuration is any of 1:1:1, 1:1:1:1, 2:1:1, or 2:2:1:1.
      7. The method of any one of embodiments 3-6, wherein in step b) the membrane configuration comprises at least one of 10 kD membranes and 30 kD membrane.
      8. The method of any one of embodiments 3-7, wherein in step b) the membrane configuration comprises at least one of narrow channel membranes and wide channel membranes.
      9. The method of any one of embodiments 1-8, wherein in step a) the polypeptide preparation is formulated in a viscosity-reducing excipient.
      10. The method of any one of embodiments 1-9, wherein in step b) the operating temperature is about 15-50° C. or about 30-50° C.
      11. The method of any one of embodiments 1-10, wherein in step b) the feed flux is about 5-25 LMH or about 25-50 LMH.
      12. The method of any one of embodiments 1-11, wherein the operating viscosity in step b) is about 200-400 cP or about 200-300 cP.
      13. The method of any one of embodiments 1-12, wherein in step b) the retentate pressure is about 5-20 psi.
      14. The method of any one of embodiments 1-13, wherein the one or more purification processes of step a) comprises an ultrafiltration.
      15. The method of any one of embodiments 1-14, wherein the one or more purification processes of step a) comprises at least two ultrafiltration operations.
      16. The method of any one of embodiments 1-15, wherein the one or more purification processes of step a) comprises a diafiltration.
      17. The method of any one of embodiments 1-16, wherein step a) comprises
i) subjecting the polypeptide preparation to an ultrafiltration operation to a first target concentration,
ii) subjecting the polypeptide preparation to a diafiltration operation for buffer exchange, and
iii) subjecting the polypeptide preparation to a second ultrafiltration operation to a second target concentration and recovering the first resulting pool having a viscosity of 5-40 cP.
   18. The method of embodiment 17, wherein the first target concentration is 5-80 g/L.
   19. The method of embodiment 17 or 18, wherein the buffer exchange is a 6-12× exchange.
   20. The method of any one of embodiments 17-19, wherein the second target concentration is 100-150 g/L or 100-200 g/L.
   21. The method of any one of embodiments 1-20, wherein the polypeptide preparation comprises the polypeptide and one or more impurities.
   22. The method of any one of embodiments 1-21, wherein the one or more purification processes of step a) comprise at least one of: a harvest step, a sample conditioning step, a viral filtration step, a viral inactivation step, a chromatography polishing step, a chromatography capture step, a dilution step, a tangential flow depth filtration step, a depth filtration step, a diafiltration step, an ultrafiltration step, a guard filtration step, a precipitation step, and a flocculation step.
   23. The method of any one of embodiments 1-22, further comprising: subjecting the second resulting pool to one or more subsequent purification processes, wherein the one or more subsequent purification processes comprise at least one of: a viral filtration step, a viral inactivation step, a chromatography polishing step, a chromatography capture step, a dilution step, a sample conditioning step, a diafiltration step, a tangential flow depth filtration step, a depth filtration step, a sterile filtration step, and a guard filtration step.
   24. A system for purifying a polypeptide from a polypeptide preparation, the system configured to perform the following steps in order:
a) subject the polypeptide preparation to one or more purification processes and recover a first resulting pool having a viscosity of about 5-40 cP; and
b) subject the pool recovered from step a) to a single pass tangential flow filtration (SPTFF) operation and recover a second resulting pool having an operating viscosity of about 41-400 cP,
wherein,
   i) the operating temperature is about 15-55° C., and
   ii) the feed flux is about 5-50 L/m²/hr (LMH).
      25. The system of embodiment 24, wherein in step a) the viscosity of the first recovered pool is 10-35 cP.
      26. The system of embodiment 24 or 25, wherein in step b) the STPFF operation comprises use of a serial membrane configuration.
      27. The system of any one of embodiments 24-26, wherein in step b) the STPFF operation comprises use of a parallel membrane configuration.
      28. The system of any one of embodiments 26-27, wherein in step b) the SPTFF operation comprises use of both a serial membrane configuration and a parallel membrane configuration.
      29. The system of any one of embodiments 26-28, wherein in step b) wherein the membrane configuration is any of 1:1:1, 1:1:1:1, 2:1:1, or 2:2:1:1.
      30. The system of any one of embodiments 26-29, wherein in step b) the membrane configuration comprises at least one of 10 kD membranes and 30 kD membrane.
      31. The system of any one of embodiments 26-30, wherein in step b) the membrane configuration comprises at least one of narrow channel membranes and wide channel membranes.
      32. The system of any one of embodiments 24-31, wherein in step a) the polypeptide preparation is formulated in a viscosity-reducing excipient.
      33. The system of any one of embodiments 24-32, wherein in step b) the operating temperature is about 15-50° C. or about 30-50° C.
      34. The system of any one of embodiments 24-33, wherein in step b) the feed flux is about 5-25 LMH or about 25-50 LMH.
      35. The system of any one of embodiments 24-34, wherein the operating viscosity in step b) is about 200-400 cP or about 200-300 cP.
      36. The system of any one of embodiments 24-35, wherein in step b) the retentate pressure is about 5-20 psi.
      37. The system of any one of embodiments 24-36, wherein the one or more purification processes of step a) comprises an ultrafiltration.
      38. The system of any one of embodiments 24-37, wherein the one or more purification processes of step a) comprises at least two ultrafiltration operations.
      39. The system of any one of embodiments 24-38, wherein the one or more purification processes of step a) comprises a diafiltration.
      40. The system of any one of embodiments 24-39, wherein in step a) the system is configured to:
i) subject the polypeptide preparation to an ultrafiltration operation to a first target concentration,
ii) subject the polypeptide preparation to a diafiltration operation for buffer exchange, and
iii) subject the polypeptide preparation to a second ultrafiltration operation to a second target concentration and recover the first resulting pool having a viscosity of 5-40 cP.
   41. The system of embodiment 40, wherein the first target concentration is 5-80 g/L.
   42. The system of embodiment 40 or 41, wherein the buffer exchange is a 6-12× exchange.
   43. The system of any one of embodiments 40-42, wherein the second target concentration is 100-150 g/L or 100-200 g/L.
   44. The system of any one of embodiments 24-43, wherein the polypeptide preparation comprises the polypeptide and one or more impurities.
   45. The system of any one of embodiments 24-44, wherein the one or more purification processes of step a) comprise at least one of: a harvest step, a sample conditioning step, a viral filtration step, a viral inactivation step, a chromatography polishing step, a chromatography capture step, a dilution step, a tangential flow depth filtration step, a depth filtration step, a diafiltration step, an ultrafiltration step, a guard filtration step, a precipitation step, and a flocculation step.
   46. The system of any one of embodiments 24-44, wherein the system is further configured to: subject the second resulting pool to one or more subsequent purification processes, wherein the one or more subsequent purification processes comprise at least one of: a viral filtration step, a viral inactivation step, a chromatography polishing step, a chromatography capture step, a dilution step, a sample conditioning step, a diafiltration step, a tangential flow depth filtration step, a depth filtration step, a sterile filtration step, and a guard filtration step.

Those skilled in the art will recognize that several embodiments are possible within the scope and spirit of the disclosure of this application. The disclosure is illustrated further by the examples below, which are not to be construed as limiting the disclosure in scope or spirit to the specific procedures described therein.

### EXAMPLES

### Example 1

This example shows a study to investigate the use of a number of membranes arranged in series for SPTFF filtration at different feed concentrations. Either three or four membranes were tested (3 stage or 4 stage) in series at feed concentrations ranging from 90 g/L to 160 g/L. PELLICON 3 membranes, 30 kD, 88 cm² and outfitted with a D screen were used. The feed contained an anti-IL13 monoclonal antibody. The feed was subjected to various purification steps prior to the SPTFF process, including: Detergent Viral Inactivation, Protein A Chromatography Capture (MabSelect™ SuRe™), Low pH Viral Inactivation, an IEX Chromatography Polishing Step, a HIC Chromatography Polishing step, a Viral Filtration step, an Ultrafiltration/Infiltration/Ultrafiltration Operation, and a Dilution Step (in formulation buffer). Results are presented in Table 3.

TABLE 3

| | | | Feed | Feed | Retentate |
|---|---|---|---|---|---|
| | Feed Conc. | Temp. | Pressure | Flux | conc. |
| Configuration | (g/L) | (° C.) | (psi) | (LMH) | (g/L) |
| 3 stage (1:1:1) | 90 | 50 | 10 | 22 | 128 |
| 4 stage (1:1:1:1) | 90 | 50 | 20 | 11 | 177 |
| 4 stage (1:1:1:1) | 125 | 50 | 35 | 7 | 233 |
| 3 stage (1:1:1) | 160 | 50 | 25 | 11 | 242 |
| 4 stage (1:1:1:1) | 160 | 50 | 40 | 5 | N/A* |

*membrane fouling

### Example 2

This example shows a study to investigate the sensitivity of SPTFF parameters. Three different feed concentrations were each tested at two temperatures. Feed flux ranged from 5-35 LMH. A three stage, 1:1:1 configuration of a PELLICON 3 membranes, 30 kD, 88 cm², outfitted with D screens were used. Outputs evaluated included feed pressure, volumetric concentration factor (VCF), and retentate concentration.

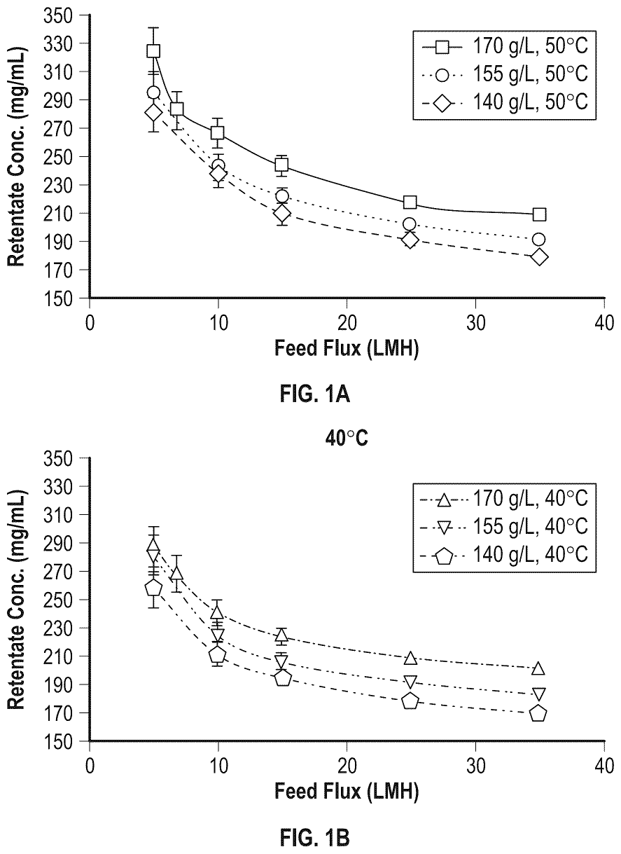

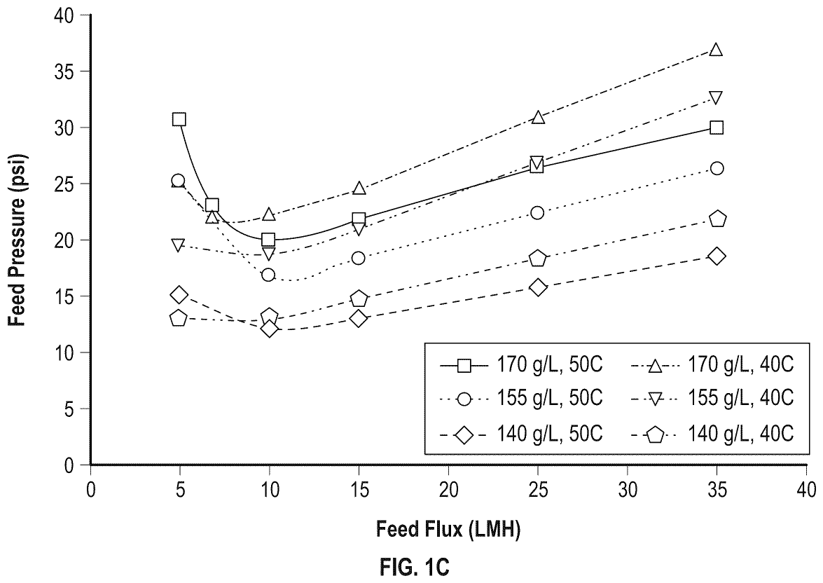

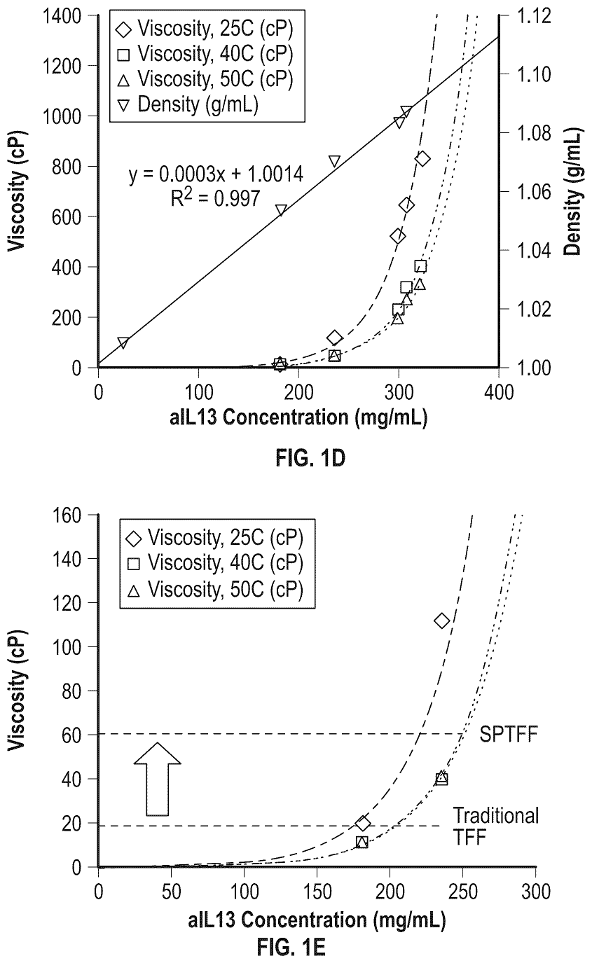

Results are shown in FIGS. 1A and 1B and in Table 4.

TABLE 4

| Temp | Feed Conc. | Feed Flux | Retentate Conc. |
|---|---|---|---|
| (° C.) | (g/L) | (LMH) | (g/L) |
| 50 | 170 | 5 | 324 |
| 50 | 140 | 10-15 | 211-238 |
| 50 | 170 | 10-15 | 243-267 |
| 40 | 140 | 10-15 | 196-211 |
| 40 | 170 | 10-15 | 224-241 |

A wide range of predictable, steady state operating conditions were screened. No membrane fouling was observed. Pools were stable throughout processing as measured by size exclusion chromatography. The pools had 1.1-1.2% high molecular weight species (HMWS) and <0.1% very high molecular weight species (vHMWS).

As shown in FIG. 1C, at high feed fluxes, flowrate-driven high pressure dominates. Minimum pressure at about 10 LMH indicates a transition to viscosity-driven resistance. A target operating range for robust processing is just below 10 LMH.

Table 5 shows the properties of high concentration anti-IL13 formulations after SPTFF. The results show that SPTFF had demonstrated steady-state operation at >300 cP. See FIG. 1D. By interpolating the retentate viscosity at minimum feed pressure, a target operating viscosity limit is estimated to be >60 cP (FIG. 1E).

TABLE 5

| | | 25° C. | 40° C. | 50° C. |
|---|---|---|---|---|
| Conc. | Density | Viscosity | Viscosity | Viscosity |
| (mg/mL) | (g/mL) | (cP) | (cP) | (cP) |
| 27 | 1.008 | N/A | N/A | N/A |
| 182 | 1.053 | 19 | 11 | 11 |
| 236 | 1.069 | 112 | 39 | 42 |
| 299 | 1.083 | 519 | 229 | 196 |
| 308 | 1.086 | 645 | 311 | 271 |
| 323 | N/A | 824 | 400 | 332 |

### Example 3

This example shows SPTFF of another antibody, Fab-conjugate aFDv3.2 following an ultrafiltration/diafiltration step. A first ultrafiltration step increased concentration from about 5 g/L to about 50 g/L, 50 g/L being the diafiltration concentration. A diafiltration step followed the first ultrafiltration step, then a second ultrafiltration step adjusted the concentration to a final concentration target of about 125 g/L.

A four stage, 1:1:1:1 configuration of a PELLICON 3 membranes, 30 kD, 88 cm², outfitted with D screens were used. The operating temperature of the SPTFF process was 40 degrees Celsius.

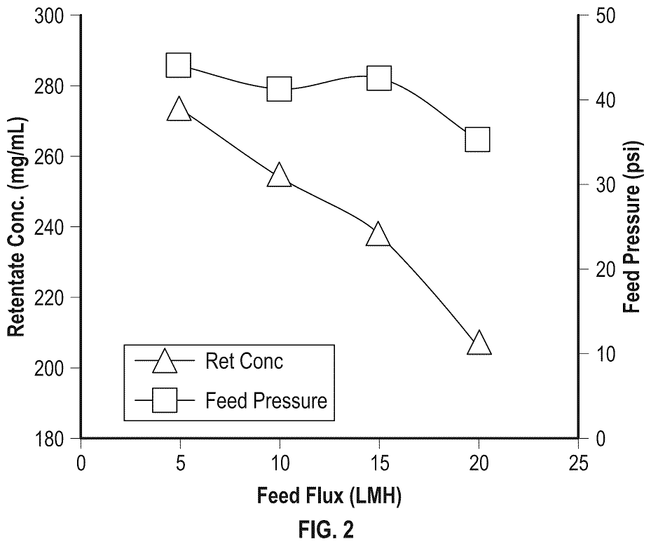

Results are shown in FIG. 2 and in Table 6.

TABLE 6

| | Retentate | Feed | | | | Retentate |
|---|---|---|---|---|---|---|
| Feed Flux | Pressure | Pressure | Delta P | TMP | | Conc. |
| (LMH) | (psi) | (psi) | (psi) | (psi) | VCF | (mg/mL) |
| 20 | 5 | 35 | 30 | 17 | 1.66 | 206 |
| 15 | 11 | 43 | 32 | 24 | 1.91 | 236 |
| 10 | 10 | 41 | 31 | 23 | 2.05 | 253 |
| 5 | 12 | 44 | 32 | 25 | 2.15 | 267 |

### Exemplary Computing Device

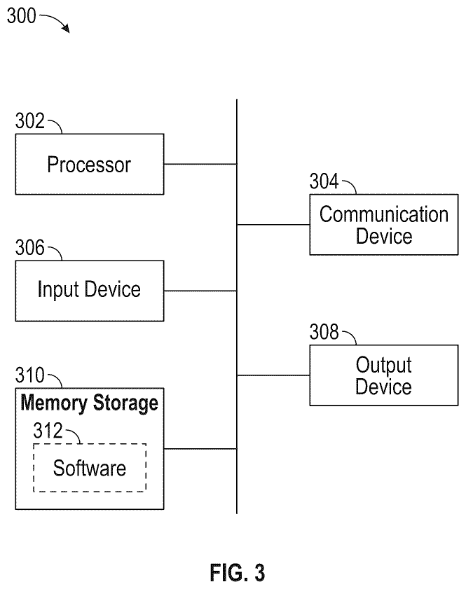

FIG. 3 depicts an exemplary computing device 300, which may be implemented in accordance with one or more examples of the disclosure. Device 300 can be a host computer connected to a network. Device 300 can be a client computer or a server. As shown in FIG. 3, device 300 can be any suitable type of microprocessor-based device, such as a personal computer, workstation, server, or handheld computing device (portable electronic device) such as a phone or tablet. The device can include, for example, one or more of processors 302, input device 306, output device 308, storage 310, and communication device 304. Input device 306 and output device 308 can generally correspond to those described above and can either be connectable or integrated with the computer.

Input device 306 can be any suitable device that provides input, such as a touch screen, keyboard or keypad, mouse, or voice-recognition device. Output device 308 can be any suitable device that provides output, such as a touch screen, haptics device, or speaker.

Storage 310 can be any suitable device that provides storage, such as an electrical, magnetic, or optical memory, including a RAM, cache, hard drive, or removable storage disk. Communication device 304 can include any suitable device capable of transmitting and receiving signals over a network, such as a network interface chip or device. The components of the computer can be connected in any suitable manner, such as via a physical bus or wirelessly.

Software 312, which can be stored in storage 310 and executed by processor 302, can include, for example, the programming that embodies the functionality of the present disclosure (e.g., as embodied in the devices as described above).

Software 312 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 310, that can contain or store programming for use by or in connection with an instruction execution system, apparatus, or device.

Software 312 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Device 300 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 300 can implement any operating system suitable for operating on the network. Software 312 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

1. A method for purifying a polypeptide from a polypeptide preparation, comprising the following steps in order:
a) subjecting the polypeptide preparation to one or more purification processes and recovering a first resulting pool having a viscosity of about 5-40 cP; and
b) subjecting the pool recovered from step a) to a single pass tangential flow filtration (SPTFF) operation and recovering a second resulting pool having an operating viscosity of about 41-400 cP,
wherein,
i) the operating temperature is about 15-55° C., and
ii) the feed flux is about 5-50 L/m²/hr (LMH).

2. The method of claim 1, wherein in step a) the viscosity of the first recovered pool is 10-35 cP.

3. The method of claim 1, wherein in step b) the STPFF operation comprises use of a serial membrane configuration.

4. The method of claim 1, wherein in step b) the STPFF operation comprises use of a parallel membrane configuration.

5. The method of claim 3, wherein in step b) the SPTFF operation comprises use of both a serial membrane configuration and a parallel membrane configuration.

6. The method of claim 3, wherein in step b) wherein the membrane configuration is any of 1:1:1, 1:1:1:1, 2:1:1, or 2:2:1:1.

7. The method of claim 3, wherein in step b) the membrane configuration comprises at least one of 10 kD membranes and 30 kD membrane.

8. The method of claim 3, wherein in step b) the membrane configuration comprises at least one of narrow channel membranes and wide channel membranes.

9. The method of claim 1, wherein in step a) the polypeptide preparation is formulated in a viscosity-reducing excipient.

10. The method of claim 1, wherein in step b) the operating temperature is about 15-50° C. or about 30-50° C.

11. The method of claim 1, wherein in step b) the feed flux is about 5-25 LMH or about 25-50 LMH.

12. The method of claim 1, wherein the operating viscosity in step b) is about 200-400 cP or about 200-300 cP.

13. The method of claim 1, wherein in step b) the retentate pressure is about 5-20 psi.

14. The method of claim 1, wherein the one or more purification processes of step a) comprises an ultrafiltration.

15. The method of claim 1, wherein the one or more purification processes of step a) comprises at least two ultrafiltration operations.

16. The method of claim 1, wherein the one or more purification processes of step a) comprises a diafiltration.

17. The method of claim 1, wherein step a) comprises
i) subjecting the polypeptide preparation to an ultrafiltration operation to a first target concentration,
ii) subjecting the polypeptide preparation to a diafiltration operation for buffer exchange, and
iii) subjecting the polypeptide preparation to a second ultrafiltration operation to a second target concentration and recovering the first resulting pool having a viscosity of 5-40 cP.

18. The method of claim 17, wherein the first target concentration is 5-80 g/L.

19. The method of claim 17, wherein the buffer exchange is a 6-12× exchange.

20. The method of claim 17, wherein the second target concentration is 100-150 g/L or 100-200 g/L.

21. The method of claim 1, wherein the polypeptide preparation comprises the polypeptide and one or more impurities.

22. The method of claim 1, wherein the one or more purification processes of step a) comprise at least one of: a harvest step, a sample conditioning step, a viral filtration step, a viral inactivation step, a chromatography polishing step, a chromatography capture step, a dilution step, a tangential flow depth filtration step, a depth filtration step, a diafiltration step, an ultrafiltration step, a guard filtration step, a precipitation step, and a flocculation step.

23. The method of claim 1, further comprising: subjecting the second resulting pool to one or more subsequent purification processes, wherein the one or more subsequent purification processes comprise at least one of: a viral filtration step, a viral inactivation step, a chromatography polishing step, a chromatography capture step, a dilution step, a sample conditioning step, a diafiltration step, a tangential flow depth filtration step, a depth filtration step, a sterile filtration step, and a guard filtration step.

24. A system for purifying a polypeptide from a polypeptide preparation, the system configured to perform the following steps in order:
a) subject the polypeptide preparation to one or more purification processes and recover a first resulting pool having a viscosity of about 5-40 cP; and
b) subject the pool recovered from step a) to a single pass tangential flow filtration (SPTFF) operation and recover a second resulting pool having an operating viscosity of about 41-400 cP,
wherein,
i) the operating temperature is about 15-55° C., and
ii) the feed flux is about 5-50 L/m²/hr (LMH).

25. The system of claim 24, wherein in step a) the viscosity of the first recovered pool is 10-35 cP.

26. The system of claim 24, wherein in step b) the STPFF operation comprises use of a serial membrane configuration.

27. The system of claim 24, wherein in step b) the STPFF operation comprises use of a parallel membrane configuration.

28. The system of claim 26, wherein in step b) the SPTFF operation comprises use of both a serial membrane configuration and a parallel membrane configuration.

29. The system of claim 26, wherein in step b) wherein the membrane configuration is any of 1:1:1, 1:1:1:1, 2:1:1, or 2:2:1:1.

30. The system of claim 26, wherein in step b) the membrane configuration comprises at least one of 10 kD membranes and 30 kD membrane.

31. The system of claim 26, wherein in step b) the membrane configuration comprises at least one of narrow channel membranes and wide channel membranes.

32. The system of claim 24, wherein in step a) the polypeptide preparation is formulated in a viscosity-reducing excipient.

33. The system of claim 24, wherein in step b) the operating temperature is about 15-50° C. or about 30-50° C.

34. The system of claim 24, wherein in step b) the feed flux is about 5-25 LMH or about 25-50 LMH.

35. The system of claim 24, wherein the operating viscosity in step b) is about 200-400 cP or about 200-300 cP.

36. The system of claim 24, wherein in step b) the retentate pressure is about 5-20 psi.

37. The system of claim 24, wherein the one or more purification processes of step a) comprises an ultrafiltration.

38. The system of claim 24, wherein the one or more purification processes of step a) comprises at least two ultrafiltration operations.

39. The system of claim 24, wherein the one or more purification processes of step a) comprises a diafiltration.

40. The system of claim 24, wherein in step a) the system is configured to:
i) subject the polypeptide preparation to an ultrafiltration operation to a first target concentration,
ii) subject the polypeptide preparation to a diafiltration operation for buffer exchange, and
iii) subject the polypeptide preparation to a second ultrafiltration operation to a second target concentration and recover the first resulting pool having a viscosity of 5-40 cP.

41. The system of claim 40, wherein the first target concentration is 5-80 g/L.

42. The system of claim 40, wherein the buffer exchange is a 6-12× exchange.

43. The system of claim 40, wherein the second target concentration is 100-150 g/L or 100-200 g/L.

44. The system of claim 24, wherein the polypeptide preparation comprises the polypeptide and one or more impurities.

45. The system of claim 24, wherein the one or more purification processes of step a) comprise at least one of: a harvest step, a sample conditioning step, a viral filtration step, a viral inactivation step, a chromatography polishing step, a chromatography capture step, a dilution step, a tangential flow depth filtration step, a depth filtration step, a diafiltration step, an ultrafiltration step, a guard filtration step, a precipitation step, and a flocculation step.

46. The system of claim 24, wherein the system is further configured to: subject the second resulting pool to one or more subsequent purification processes, wherein the one or more subsequent purification processes comprise at least one of: a viral filtration step, a viral inactivation step, a chromatography polishing step, a chromatography capture step, a dilution step, a sample conditioning step, a diafiltration step, a tangential flow depth filtration step, a depth filtration step, a sterile filtration step, and a guard filtration step.
